(19) United States
(12) Patent Application Publication
(10) Pub. No.: US 2024/0077643 A1
(43) Pub. Date: Mar. 7, 2024

(54) EVALUATING AIR QUALITY

(71) Applicant: Ambient Ridge, Inc., Mt. Kisco, NY (US)

(72) Inventor: Christopher KOH, Tuscon, AZ (US)

(73) Assignee: Ambient Ridge, Inc., Mt. Kisco, NY (US)

(21) Appl. No.: 18/458,117

(22) Filed: Aug. 29, 2023

Related U.S. Application Data
(60) Provisional application No. 63/374,517, filed on Sep. 2, 2022.

(51) Int. Cl. G01W 1/00

(52) U.S. Cl. CPC G01W 1/00

(57) ABSTRACT

In a computer-implemented method for evaluating air quality, air quality information is received for a plurality of locations for a plurality of days over a time period. For each location of the plurality of locations a severe air quality percentile for the air quality information for each location of the plurality of locations for the time period is determined and a variance of the air quality information for each location of the plurality of locations for the time period is determined. The plurality of locations is evaluated according to the severe air quality percentile for the air quality information and the variance of the air quality information for each location.

### RELATED APPLICATION

This application claims priority to and the benefit of co-pending U.S. Provisional Patent Application 63/374,517, filed on Sep. 2, 2022, entitled “AIR QUALITY EVALUATION,” by Christpher Koh, having Attorney Docket No. AR-002.PRO, and assigned to the assignee of the present application, which is incorporated herein by reference in its entirety.

### BACKGROUND

Environmental and natural hazard information is important for allowing individuals, property developers and owners, as well as renters, to know and understand the climate and environmental hazard and risk information associated with the locations. There are a myriad of sources for obtaining certain types of environmental and natural hazard information, such as government institutions at the national, regional, and local level, as well as private organizations. This information may not be in a consumer digestible format that is decipherable to users in a way that provides useful information to users for evaluating climate and environmental hazards and risks for a location.

### BRIEF DESCRIPTION OF DRAWINGS

The accompanying drawings, which are incorporated in and form a part of the Description of Embodiments, illustrate various embodiments of the subject matter and, together with the Description of Embodiments, serve to explain principles of the subject matter discussed below. Unless specifically noted, the drawings referred to in this Brief Description of Drawings should be understood as not being drawn to scale. Herein, like items are labeled with like item numbers.

FIG. 1 is a block diagram illustrating an embodiment of an example system for transforming and searching environmental hazard and risk information, according to embodiments.

FIG. 2 is a block diagram illustrating an example environmental hazard and risk data transformation module, according to an embodiment.

FIG. 3 is a block diagram illustrating an example air quality evaluation, according to an embodiment.

FIG. 4 illustrates an example visualization of the air quality metric described herein, according to embodiments.

FIG. 5 illustrates an example visualization of the ozone only air quality metric described herein, according to embodiments.

FIG. 6 is a block diagram of an example computer system upon which embodiments of the present invention can be implemented.

FIG. 7 illustrates a flow diagram illustrating an example method for evaluating air quality, in accordance with embodiments.

### DESCRIPTION OF EMBODIMENTS

Reference will now be made in detail to various embodiments of the subject matter, examples of which are illustrated in the accompanying drawings. While various embodiments are discussed herein, it will be understood that they are not intended to be limited to these embodiments. On the contrary, the presented embodiments are intended to cover alternatives, modifications and equivalents, which may be included within the spirit and scope the various embodiments as defined by the appended claims. Furthermore, in this Description of Embodiments, numerous specific details are set forth in order to provide a thorough understanding of embodiments of the present subject matter. However, embodiments may be practiced without these specific details. In other instances, well known methods, procedures, components, and circuits have not been described in detail as not to unnecessarily obscure aspects of the described embodiments.

Some portions of the detailed descriptions which follow are presented in terms of procedures, logic blocks, processing and other symbolic representations of operations on data bits within a computer memory. These descriptions and representations are the means used by those skilled in the data processing arts to most effectively convey the substance of their work to others skilled in the art. In the present application, a procedure, logic block, process, or the like, is conceived to be one or more self-consistent procedures or instructions leading to a desired result. The procedures are those requiring physical manipulations of physical quantities. Usually, although not necessarily, these quantities take the form of electrical or magnetic signals capable of being stored, transferred, combined, compared, and otherwise manipulated in an electronic device.

It should be borne in mind, however, that all of these and similar terms are to be associated with the appropriate physical quantities and are merely convenient labels applied to these quantities. Unless specifically stated otherwise as apparent from the following discussions, it is appreciated that throughout the description of embodiments, discussions utilizing terms such as “receiving,” “determining,” “evaluating,” “performing,” “displaying,” “identifying,” “comparing,” “generating,” “executing,” “configuring,” “storing,” “directing,” “accessing,” “updating,” “collecting,” or the like, refer to the actions and processes of an electronic computing device or system such as: a host processor, a processor, a memory, a cloud-computing environment, a hyper-converged appliance, a software defined network (SDN) manager, a system manager, a virtualization management server or a virtual machine (VM), among others, of a virtualization infrastructure or a computer system of a distributed computing system, or the like, or a combination thereof. The electronic device manipulates and transforms data represented as physical (electronic and/or magnetic) quantities within the electronic device's registers and memories into other data similarly represented as physical quantities within the electronic device's memories or registers or other such information storage, transmission, processing, or display components.

Embodiments described herein may be discussed in the general context of processor-executable instructions or code residing on some form of non-transitory processor-readable medium, such as program modules, executed by one or more computers or other devices. Generally, program modules include routines, programs, objects, components, data structures, etc., that perform particular tasks or implement particular abstract data types. The functionality of the program modules may be combined or distributed as desired in various embodiments.

In the figures, a single block may be described as performing a function or functions; however, in actual practice, the function or functions performed by that block may be performed in a single component or across multiple components, and/or may be performed using hardware, using software, or using a combination of hardware and software. To clearly illustrate this interchangeability of hardware and software, various illustrative components, blocks, modules, circuits, and steps have been described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example mobile electronic device described herein may include components other than those shown, including well-known components.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium comprising instructions that, when executed, perform one or more of the methods described herein. The non-transitory processor-readable data storage medium may form part of a computer program product, which may include packaging materials.

The non-transitory processor-readable storage medium may include random access memory (RAM) such as synchronous dynamic random access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, and/or executed by a computer or other processor.

The various illustrative logical blocks, modules, code and instructions described in connection with the embodiments disclosed herein may be executed by one or more processors, such as one or more motion processing units (MPUs), sensor processing units (SPUs), host processor(s) or core(s) thereof, digital signal processors (DSPs), general purpose microprocessors, application specific integrated circuits (ASICs), application specific instruction set processors (ASIPs), field programmable gate arrays (FPGAs), or other equivalent integrated or discrete logic circuitry. The term “processor,” as used herein may refer to any of the foregoing structures or any other structure suitable for implementation of the techniques described herein. In addition, in some aspects, the functionality described herein may be provided within dedicated software modules or hardware modules configured as described herein. Also, the techniques could be fully implemented in one or more circuits or logic elements. A general purpose processor may be a microprocessor, but in the alternative, the processor may be any conventional processor, controller, microcontroller, or state machine. A processor may also be implemented as a combination of computing devices, e.g., a combination of an SPU/MPU and a microprocessor, a plurality of microprocessors, one or more microprocessors in conjunction with an SPU core, MPU core, or any other such configuration.

### Overview of Discussion

Embodiments described herein provide a novel process for evaluating air quality for a location. The standardized tool of choice for measuring air quality is the United States Environmental Protection Agency's AirNow air quality index (AQI). Unfortunately, daily fluctuations in AQI make understanding an area's general air quality difficult. It is tempting to use the average of recorded AQI values to do so, but average AQI may not be a reliable indicator on the health impact of air quality. For instance, a small number of days exhibiting a very high AQI may have a significant impact on an individual's health. For example, average AQI does not provide insight into the number or percentage of days having a high AQI or how prone or susceptible a location is to days having a high AQI, where a high AQI is generally defined as an AQI that is dangerous or unhealthy to exposed persons.

Embodiments described herein provide a method for evaluating air quality. Air quality information (e.g., the EPA's AQI index) is received for a plurality of locations for a plurality of days over a time period. For each location of the plurality of locations a severe air quality percentile (e.g., 90^{th} percentile AQI) for the air quality information for each location of the plurality of locations for the time period is determined and a variance of the air quality information for each location of the plurality of locations for the time period is determined. In some embodiments, a logarithm of the variance of the air quality information for each location of the plurality of locations for the time period is determined.

The plurality of locations is evaluated according to the severe air quality percentile for the air quality information and the variance of the air quality information for each location. In some embodiments, evaluating the plurality of locations includes performing a maximum/minimum normalization operation on the severe air quality percentile for the air quality information and the variance of the air quality information for each location of the plurality of locations. In some embodiments, evaluating the plurality of locations includes performing a clustering operation on the severe air quality percentile for the air quality information and the variance of the air quality information for each location of the plurality of locations. For example, the clustering operation can determine five clusters of locations that are indicative of the relative air quality for the clustered locations (e.g., air quality that is great, good, average, poor, very poor). In some embodiments, a visualization based at least in part on the evaluation of the plurality of locations according to the severe air quality percentile for the air quality information and the variance of the air quality information is displayed.

In some described embodiments, a process for generating an air quality metric (also referred to herein as the “AreaAir index”) is provided. The described air quality metric makes sense of such daily fluctuations by considering a severe air quality percentile (e.g., 90th percentile AQI) and the logarithm of the variance (labeled in the graphic as susceptance to short term high AQI) of recorded AQI values for a period of time (e.g., a year). It should be appreciated that the 90th percentile AQI value can be any percentile, where the 90th percentile is used as an example that is representative of negative health impacts both in areas having very severe and prolonged high AQI values and areas without such extremes. The variance of the daily AQI based off the EPA's maximum value reported AQI for that day is used to encapsulate the frequency and severity of high AQI days in order to take into account the negative effects of short term exposure to high air pollution (which would not show up on the 90th percentile metric). Additionally, areas prone to pronounced seasonal changes in AQI will also have high variance scores.

In some embodiments, location features are then maximum-minimum normalized on the interval [0,1] for grouping by a Gaussian mixture clustering algorithm (e.g., implemented in the sklearn Python library). The clustering algorithm is used to generate a predetermined number of clusters (e.g., five or six clusters) of different air quality severity. In some embodiments, at least one cluster identifies a group of locations that has a moderate 90^{th} percentile AQI, but a high variance. It should be appreciated that similar methodologies can be applied to other environmental data, such as ozone. The clustering gives results in a number of groups of varying AQI severity that can be visualized and color-coded.

### Example System for Transformation of Inconsistent Environmental and Natural Hazard Data

Example embodiments described herein provide systems and methods for generating accessible and easy to understand information from data sources that are often inconsistent and disparate. The data, coming from disparate sources and in different types, is transformed into consistent data that can be compared and analyzed appropriately in a normalized fashion. This search data can be customized according to search preferences, to provide an improved and enhanced user experience.

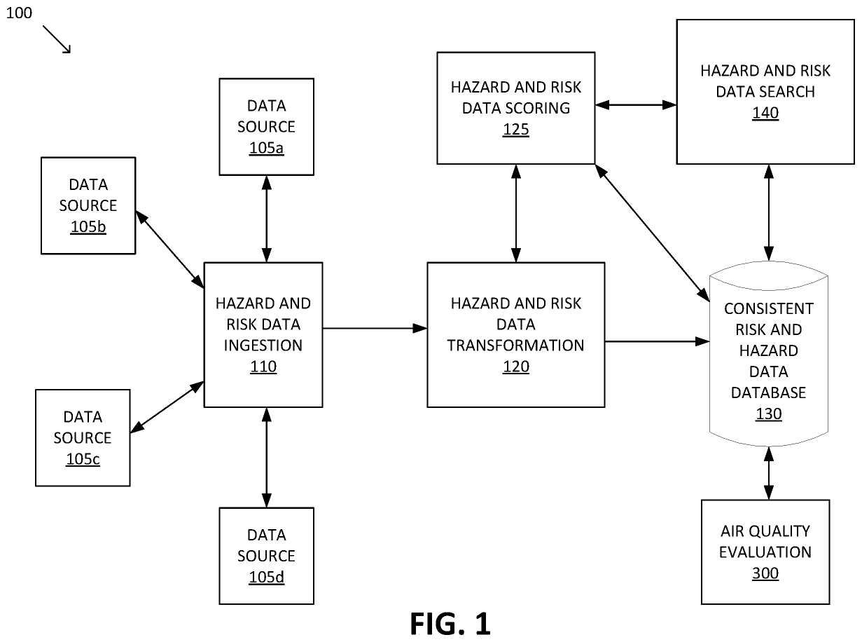

FIG. 1 is a block diagram illustrating an embodiment of an example system 100 for transforming and searching environmental hazard and risk information, according to embodiments. System 100 includes hazard and risk data ingestion module 110 for ingesting data from disparate data source 105a thorough 105d, hazard and risk data transformation module 120, hazard and risk data scoring 125, consistent hazard and risk data database 130, and hazard and risk data search module 140. It should be appreciated that hazard and risk data ingestion module 110, hazard and risk data transformation module 120, hazard and risk data scoring 125, consistent hazard and risk data database 130, and hazard and risk data search module 140 can be under the control of a single component of an enterprise computing environment (e.g., a computer system 600) or can be distributed over multiple components. In some embodiments, system 100 includes air quality evaluation module 300 for evaluating air quality for a plurality of locations.

It should be appreciated that system 100 may ingest data at hazard and risk data ingestion module 110 from a variety of sources, including open data sources such as federal government databases, e.g., the Environmental Protection Agency (EPA) or the National Oceanic and Atmospheric Administration (NOAA), as well as state, local city, county and other databases.

In accordance with some embodiments, hazard data is requested from data sources 105a-d. For example, a CRON Based Lambda Function that runs periodically (e.g., daily) makes an HTTP POST Request to a data source 105a. For example, an HTTP Post Request can be made to an EPA Facility Registry Service (FRS) MapServer to request particular information. In a specific example, the request can be for information marked “ACRES” to identify brownfield locations. In some embodiments, the data received is reconciled against stored data to determine whether new data is received. If there is no new data received after comparison to the stored data, the process completes. If new data is identified, the data is forwarded to hazard and risk data transformation module 120.

The data is received at hazard and risk data transformation module 120 and, coming from disparate sources and in different types, is transformed into consistent data that can be compared and analyzed appropriately in a normalized fashion. The consistent data is stored at consistent hazard and risk data database 130. Hazard and risk data search module 140 is configured to receive and perform searches on the data of consistent hazard and risk data database 130.

Conventional environmental and natural hazard information is typically varied and complex in terms of data source, data type, and data formats, such that the data is inconsistent across different sources, making comparison generally unachievable across different sources. The underlying data for these types of data can be particularly challenging. These challenges include:
the difficulty in locating or accessing certain data;
the fragmentation of the data (in some cases with respect to the same hazard and in other cases across hazards);
the inconsistency with which that data is presented (in some cases with respect to the same hazard and in other cases across hazards);
how technical or scientific the information is where available, making it hard to understand or interpret for the average consumer; and
the different frequencies with which the datasets update (giving rise to different “pull” frequencies).

The described embodiments address these challenges, enabling the ingestion of relevant environmental health and natural hazards' or potential risks' information and produce meaningful reports. In order to allow comparisons and analyses of such data, embodiments described herein transform the data to provide standardized data that is capable of being compared.

After the data has been accessed and ingested, the system is configured to transform the data by standardizing or normalizing the data, and aggregating the data to prepare the data for the geospatial, scoring, weighting and selection innovations designed to enable the platform's features.

The system ingests and then transforms a range of types of data, much of which is environmental health and natural hazard data with inconsistency challenges as described above, pertaining to various areas across a region (e.g., the United States) to provide consistency or compatibility to that data:
1. presented on geomaps for the entire geographic space for which the data is received;
2. with usable interfaces created or designed to provide the information in ways to make it more understandable and accessible; and
3. then processes to standardize and/or normalize the data across the region such that area risk data is able to be transposed onto geomaps and to be subject to processing search, scoring, comparing and/or weighing the data.

Because this integrated data is often not “clean” data, significant standardization and/or normalization work is often necessary in addition to reconciliation work to prepare the data and to verify its integrity as it is ingested and then integrated onto the environmental and natural hazards intelligence platform and database.

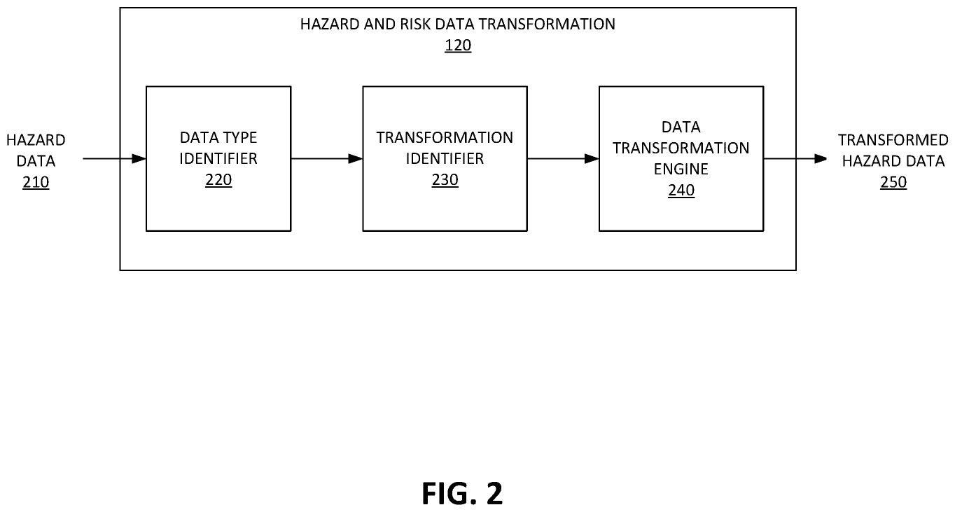

FIG. 2 is a block diagram illustrating an example environmental hazard and risk data transformation module 120, according to an embodiment. Environmental hazard and risk data transformation module 120 includes data type identifier 220, transformation identifier 230, and data transformation engine 240. It should be appreciated that data type identifier 220, transformation identifier 230, and data transformation engine 240 can be under the control of a single component of an enterprise computing environment (e.g., a computer system 600) or can be distributed over multiple components.

Hazard data 210 is received (e.g., from hazard and risk data ingestion module 110) at data type identifier 220 of environmental hazard and risk data transformation module 120. Data type identifier 220 is configured to inspect hazard data 210 and to determine a data type of hazard data 210. For example, data received from an EPA Facility Registry Service (FRS) MapServer may be received in a GeoJSON format (e.g., to describe brownfield locations). The data is further inspected at transformation identifier 230 to determine what type of transformation or transformations to apply to hazard data 210 upon identification of the data type.

At data transformation engine 240, hazard data 210 is transformed according to the transformation or transformations identified at transformation identifier 230. For example, transformations to hazard data 210 can include: renaming object keys (e.g., changing facility_name to name), changing geospatial projections (e.g., transforming EPSG:4269 data format to EPSG:4326 data format), transforming GeoJSON results into a standardized JSON format, etc. Data transformation engine 240 generates transformed hazard data 250, and forwards transformed hazard data 250 to consistent hazard and risk data database 130 for storage. For example, transformed hazard data 250 is forwarded as a GraphQL Mutation to the consistent hazard and risk data database 130. In some embodiments, consistent hazard and risk data database 130 is a geographic information system (GIS) database.

In some embodiments, concurrent or subsequent the generation of transformed hazard data 250, hazard and risk data scoring 125 performs an area-based scoring operation on the transformed hazard data 250. Performing the area-based scoring operation at this point allows for the precomputation and storage of the precomputed scores, that can ultimately be returned responsive to search request. This is of particular advantage for large and dynamic datasets, such as those pertaining to air quality index (AQI), so as to provide a fast response time. In some embodiments, data sets having less data (e.g., brownfields or nuclear plants) can be computed at request time. It should be appreciated that the scoring operation can be performed at search time or at ingestion, and that the precomputation allows for the reduction of computational resources used at the time of the search.

Scoring operations are applied to the hazard data (e.g., transformed hazard data) to provide information of the relative risk associated with particular hazards. The scoring operations are applied to an area, also referred to herein as a geozone. In accordance with some embodiments, the geozone based scoring operation appends locations with geozone based datasets (e.g., counties, zip codes, census tracts, or any other polygon based feature).

Locations are appended with geozone based datasets (e.g., counties, zip codes, census tracts, or any other polygon based feature). Various operations can be used to append the locations using different operations, such as and without limitation: Overlapping Hierarchical Clustering (OHC), DBScan, and K-means analysis. New densities (e.g., of brownfields) are applied within the geozones as parameters to the scoring algorithm which precomputes a score. It should be appreciated that these operations generally associate risks and hazards, and the scores thereof, to geographic regions (e.g., geozones).

Hazard and risk data scoring 125 forwards the scoring information to consistent hazard and risk data database 130 for storage along with the associated hazard data. For example, the scoring information is forwarded as a GraphQL Mutation to the consistent hazard and risk data database 130.

### Example System for Evaluating Air Quality

Embodiments described herein provide a novel process for evaluating air quality for a location. The standardized tool of choice for measuring air quality is the United State Environmental Protection Agency's AirNow air quality index (AQI). Unfortunately, daily fluctuations in AQI make understanding an area's general air quality difficult. It is tempting to use the average of recorded AQI values to do so, but average AQI may not be a reliable indicator on the health impact of air quality. For instance, a small number of days exhibiting a very high AQI may have significant impact on an individual's health. For example, average AQI does not provide insight into the number or percentage of days having a high AQI or how prone or susceptible a location is to days having a high AQI, where a high AQI is generally defined as an AQI that is dangerous or unhealthy to exposed persons.

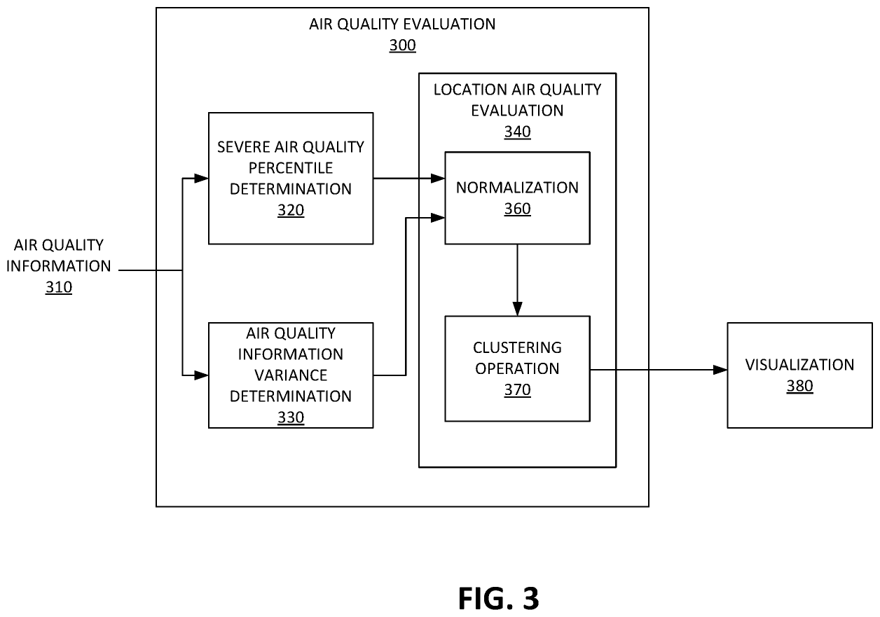

FIG. 3 is a block diagram illustrating an example air quality evaluation system 300, according to an embodiment. Air quality evaluation system 300 includes severe air quality percentile determination module 320, air quality information variance determination module 330, and location air quality evaluation module 340. It should be appreciated that severe air quality percentile determination module 320, air quality information variance determination module 330, and location air quality evaluation module 340, can be under the control of a single component of an enterprise computing environment (e.g., a computer system 600) or can be distributed over multiple components.

As utilized herein, air quality index (AQI) refers to the air quality index utilized by the United States Environmental Protection Agency (EPA) for reporting air quality. It should be appreciated that other air quality indices are measurements may be utilized in accordance with the described embodiments, and that air quality information as utilized herein can include AQI or other air quality metrics or indices.

With reference to FIG. 3, air quality information 310 (e.g., AQI) is received for a plurality of locations at air quality evaluation system 300. It should be appreciated that air quality information 310 can be received from a remote source (e.g., from an EPA data source) or from a database associated with air quality evaluation system 300 (e.g., consistent hazard and risk data database 130). Air quality information 310 is forwarded to severe air quality percentile determination module 320, air quality information variance determination module 330 for processing. It should be appreciated that air quality information 310 can include data for air quality that spans a time period (e.g., a year). In some embodiments, air quality information 310 includes daily readings of AQI for each day, or other periodic instances (e.g., every other day) over the time period. It should be appreciated that the air quality information should be received for approximately half of the days of the time period is received for each location of the plurality of locations (e.g., at least 180 days of year).

Severe air quality percentile determination module 320 is configured to determine a severe air quality percentile for the air quality information for each location of the plurality of locations for the time period. For example, the severe air quality percentile can be the 90^{th} percentile AQI for a location. It should be appreciated that the 90th percentile AQI value can be any percentile, where the 90th percentile is used as an example that is representative of negative health impacts both in areas having very severe and prolonged high AQI values and areas without such extremes. Other percentile values (e.g., 85^{th} percentile or 95^{th} percentile) can also be used in accordance with the described embodiments. The severe air quality percentile for each location is forwarded to location air quality evaluation module 340 for evaluation.

Air quality information variance determination module 330 is configured to determine a variance of the air quality information for each location of the plurality of locations for the time period is determined. In some embodiments, a logarithm of the variance of the air quality information for each location of the plurality of locations for the time period is determined. For example, the variance of the daily AQI based off the EPA's maximum value reported AQI for that day is used to encapsulate the frequency and severity of high AQI days in order to take into account the negative effects of short term exposure to high air pollution (which would not show up on the severe air quality percentile determination). Additionally, areas prone to pronounced seasonal changes in AQI will also have high variance scores. The air quality information variance for each location is forwarded to location air quality evaluation module 340 for evaluation.

The plurality of locations is evaluated according to the severe air quality percentile for the air quality information and the variance of the air quality information for each location. The severe air quality percentile for each location and the air quality information variance for each location are received at location air quality evaluation module 340 for evaluation.

In some embodiments, the severe air quality percentile for each location and the air quality information variance for each location are received at normalization module 360 that performs a maximum/minimum normalization operation on the severe air quality percentile for the air quality information and the variance of the air quality information for each location of the plurality of locations. In some embodiments, location features are maximum/minimum normalized on the interval [0,1].

In some embodiments, the severe air quality percentile for the air quality information and the variance of the air quality information for each location of the plurality of locations are received at clustering operation module 370. In some embodiments, the normalized values of the severe air quality percentile for the air quality information and the variance of the air quality information for each location of the plurality of locations are received at clustering operation module 370. In some embodiments, the clustering operation is a Gaussian mixture clustering algorithm (e.g., implemented in the sklearn Python library). However, it should be appreciated that different clustering operations can be performed.

For example, clustering operation module 370 can determine five clusters of locations that are indicative of the relative air quality for the clustered locations (e.g., air quality that is great, good, average, poor, very poor). In some embodiments, at least one cluster identifies a group of locations that has a moderate severe air quality percentile, but a high variance, which may be indicative of seasonal air quality changes. In some embodiments, the output of clustering operation module 370 is forwarded to visualization module 380.

Visualization module 380 is configured to provide a visualization based at least in part on the evaluation of the plurality of locations according to the severe air quality percentile for the air quality information and the variance of the air quality information to be displayed. In some embodiments, the clusters are associated with labels identifying the air quality for locations of each cluster (e.g., air quality that is great, good, average, poor, very poor). The clustering operation provides results in a number of groups of varying air quality severity that can be visualized and color-coded.

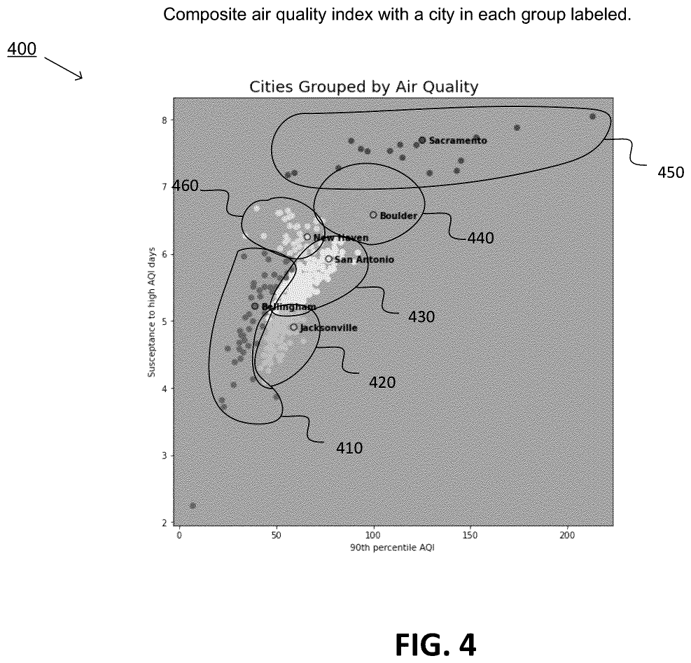

FIG. 4 illustrates an example visualization 400 of the air quality metric described herein, according to embodiments. FIG. 4 illustrates a composite air quality index visualization 400 where each dot represents a city and with a city in each group labeled, where there are six clusters (e.g., clusters 410, 420, 430, 440, 450, and 460) ranging from least severe (e.g., cluster 410) to most severe (e.g., cluster 450). Cluster 460 identifies a group of locations that has a moderate severe air quality percentile, but a high variance, which may be indicative of seasonal air quality changes. In accordance with various embodiments, the dots of cluster can be visualized using a different color, such as cluster 410 comprising dark green dots, cluster 420 comprising light green dots, cluster 430 comprising yellow dots, cluster 440 comprising orange dots, cluster 450 comprising red dots, and cluster 460 comprising pink dots. The clusters can also have associated labels, such as “great” (cluster 410), “good” (cluster 420), “average” (cluster 430), “poor” (cluster 440), “very poor” (cluster 450), and “moderate but high variance” (cluster 460). As illustrated in FIG. 4, the clustering gives rise to six groups of increasing severity from green to red, and pink (cluster 460) which has moderate 90th percentile AQI but high variance. These locations are more susceptible to high pollution days and seasonal pollution. It should be appreciated that any number of clusters, and any different combination of colors and/or labels, can be used in air quality index visualization 400.

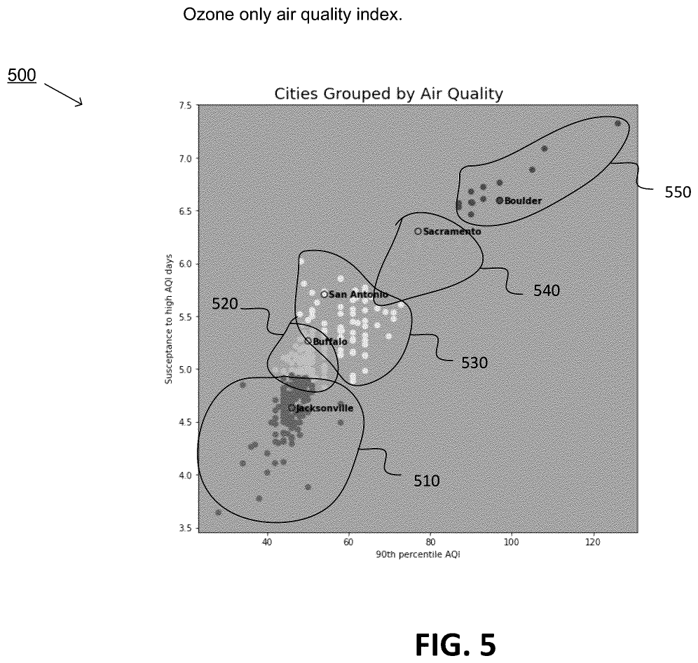

FIG. 5 illustrates an example visualization 500 of the ozone only air quality metric described herein, according to embodiments. FIG. 5 illustrates an ozone only air quality index visualization 500 where each dot represents a city, where there are five clusters (e.g., clusters 510, 520, 530, 540, and 550) ranging from least severe (e.g., cluster 510) to most severe (e.g., cluster 550). In accordance with various embodiments, the dots of cluster can be visualized using a different color, such as cluster 510 comprising dark green dots, cluster 520 comprising light green dots, cluster 530 comprising yellow dots, cluster 540 comprising orange dots, and cluster 550 comprising red dots. The clusters can also have associated labels, such as “great” (cluster 510), “good” (cluster 520), “average” (cluster 530), “poor” (cluster 540), and “very poor” (cluster 550). As illustrated in FIG. 5, the clustering gives rise to five groups of increasing severity from dark green to red. It should be appreciated that any number of clusters, and any different combination of colors and/or labels, can be used in air quality index visualization 500.

The described embodiments provide a process for generating an air quality metric that evaluates the AQI values (e.g., reported to the Environmental Protection Agency (EPA)) according to two metrics: the 90^{th} (or other) percentile AQI that is representative of the severity and frequency of a subset of substandard AQI days that may be dangerous to the health of persons in the location, and the variance which captures how prone a location is to short term very high AQI days.

### Example Computer System

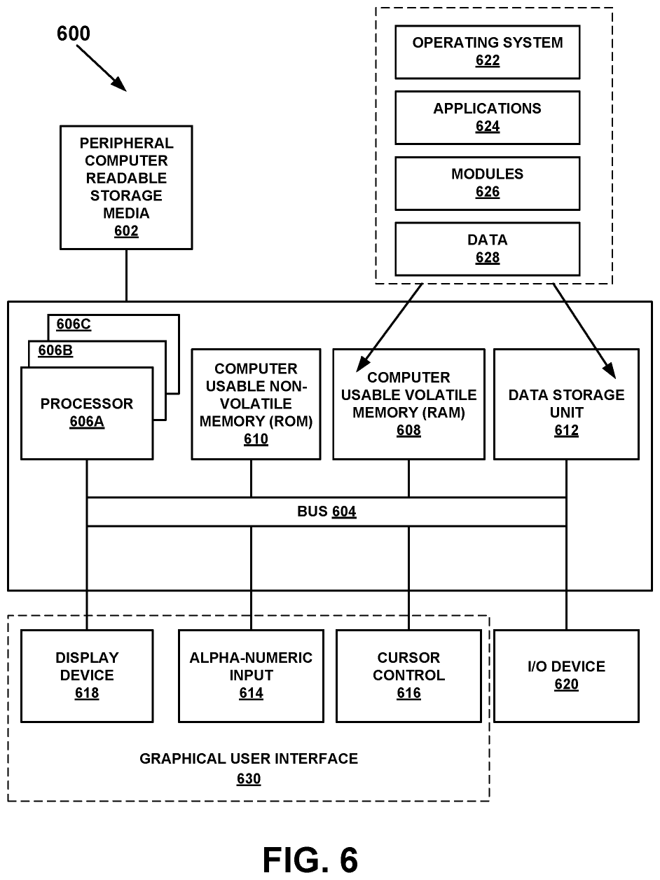

FIG. 6 is a block diagram of an example computer system 600 upon which embodiments of the present invention can be implemented. FIG. 6 illustrates one example of a type of computer system 600 (e.g., a computer system) that can be used in accordance with or to implement various embodiments which are discussed herein.

It is appreciated that computer system 600 of FIG. 6 is only an example and that embodiments as described herein can operate on or within a number of different computer systems including, but not limited to, general purpose networked computer systems, embedded computer systems, mobile electronic devices, smart phones, server devices, client devices, various intermediate devices/nodes, standalone computer systems, media centers, handheld computer systems, multi-media devices, and the like. In some embodiments, computer system 600 of FIG. 6 is well adapted to having peripheral tangible computer-readable storage media 602 such as, for example, an electronic flash memory data storage device, a floppy disc, a compact disc, digital versatile disc, other disc based storage, universal serial bus “thumb” drive, removable memory card, and the like coupled thereto. The tangible computer-readable storage media is non-transitory in nature.

Computer system 600 of FIG. 6 includes an address/data bus 604 for communicating information, and a processor 606A coupled with bus 604 for processing information and instructions. As depicted in FIG. 6, computer system 600 is also well suited to a multi-processor environment in which a plurality of processors 606A, 606B, and 606C are present. Conversely, computer system 600 is also well suited to having a single processor such as, for example, processor 606A. Processors 606A, 606B, and 606C may be any of various types of microprocessors. Computer system 600 also includes data storage features such as a computer usable volatile memory 608, e.g., random access memory (RAM), coupled with bus 604 for storing information and instructions for processors 606A, 606B, and 606C. Computer system 600 also includes computer usable non-volatile memory 610, e.g., read only memory (ROM), coupled with bus 604 for storing static information and instructions for processors 606A, 606B, and 606C. Also present in computer system 600 is a data storage unit 612 (e.g., a magnetic or optical disc and disc drive) coupled with bus 604 for storing information and instructions. Computer system 600 also includes an alphanumeric input device 614 including alphanumeric and function keys coupled with bus 604 for communicating information and command selections to processor 606A or processors 606A, 606B, and 606C. Computer system 600 also includes a cursor control device 616 coupled with bus 604 for communicating user input information and command selections to processor 606A or processors 606A, 606B, and 606C. In one embodiment, computer system 600 also includes a display device 618 coupled with bus 604 for displaying information.

Referring still to FIG. 6, display device 618 of FIG. 6 may be a liquid crystal device (LCD), light emitting diode display (LED) device, cathode ray tube (CRT), plasma display device, a touch screen device, or other display device suitable for creating graphic images and alphanumeric characters recognizable to a user. Cursor control device 616 allows the computer user to dynamically signal the movement of a visible symbol (cursor) on a display screen of display device 618 and indicate user selections of selectable items displayed on display device 618. Many implementations of cursor control device 616 are known in the art including a trackball, mouse, touch pad, touch screen, joystick or special keys on alphanumeric input device 614 capable of signaling movement of a given direction or manner of displacement. Alternatively, it will be appreciated that a cursor can be directed and/or activated via input from alphanumeric input device 614 using special keys and key sequence commands. Computer system 600 is also well suited to having a cursor directed by other means such as, for example, voice commands. In various embodiments, alphanumeric input device 614, cursor control device 616, and display device 618, or any combination thereof (e.g., user interface selection devices), may collectively operate to provide a graphical user interface (GUI) 630 under the direction of a processor (e.g., processor 606A or processors 606A, 606B, and 606C). GUI 630 allows user to interact with computer system 600 through graphical representations presented on display device 618 by interacting with alphanumeric input device 614 and/or cursor control device 616.

Computer system 600 also includes an I/O device 620 for coupling computer system 600 with external entities. For example, in one embodiment, I/O device 620 is a modem for enabling wired or wireless communications between computer system 600 and an external network such as, but not limited to, the Internet. In one embodiment, I/O device 620 includes a transmitter. Computer system 600 may communicate with a network by transmitting data via I/O device 620.

Referring still to FIG. 6, various other components are depicted for computer system 600. Specifically, when present, an operating system 622, applications 624, modules 626, and data 628 are shown as typically residing in one or some combination of computer usable volatile memory 608 (e.g., RAM), computer usable non-volatile memory 610 (e.g., ROM), and data storage unit 612. In some embodiments, all or portions of various embodiments described herein are stored, for example, as an application 624 and/or module 626 in memory locations within RAM 608, computer-readable storage media within data storage unit 612, peripheral computer-readable storage media 602, and/or other tangible computer-readable storage media.

### Example Methods of Operation

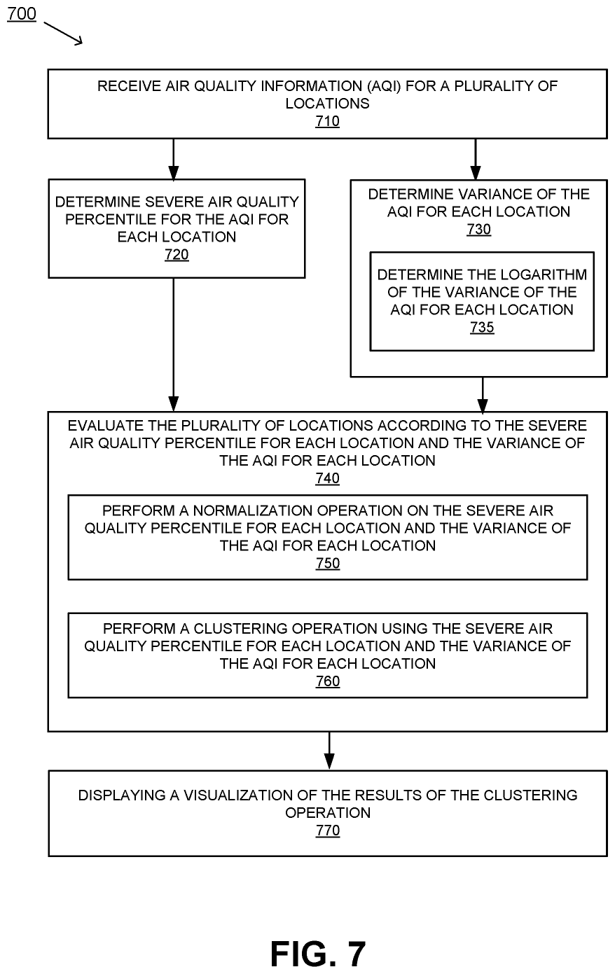

The following discussion sets forth in detail the operation of some example methods of operation of embodiments. With reference to FIG. 7, flow diagram 700 illustrates example procedures used by various embodiments. The flow diagram 700 includes some procedures that, in various embodiments, are carried out by a processor under the control of computer-readable and computer-executable instructions. In this fashion, procedures described herein and in conjunction with the flow diagrams are, or may be, implemented using a computer, in various embodiments. The computer-readable and computer-executable instructions can reside in any tangible computer readable storage media. Some non-limiting examples of tangible computer readable storage media include random access memory, read only memory, magnetic disks, solid state drives/“disks,” and optical disks, any or all of which may be employed with computer environments (e.g., computer system 600). The computer-readable and computer-executable instructions, which reside on tangible computer readable storage media, are used to control or operate in conjunction with, for example, one or some combination of processors of the computer environments and/or virtualized environment. It is appreciated that the processor(s) may be physical or virtual or some combination (it should also be appreciated that a virtual processor is implemented on physical hardware). Although specific procedures are disclosed in the flow diagram, such procedures are examples. That is, embodiments are well suited to performing various other procedures or variations of the procedures recited in the flow diagram. Likewise, in some embodiments, the procedures in flow diagram 700 may be performed in an order different than presented and/or not all of the procedures described in flow diagram 700 may be performed. It is further appreciated that procedures described in flow diagram 700 may be implemented in hardware, or a combination of hardware with firmware and/or software provided by computer system 600.

FIG. 7 depicts a flow diagram 700 for transformation of inconsistent environmental data, according to an embodiment. At procedure 710 of flow diagram 700, air quality information (e.g., the EPA's AQI index) is received for a plurality of locations for a plurality of days over a time period. As shown at procedure 720, a severe air quality percentile (e.g., 90^{th} percentile AQI) for the air quality information for each location of the plurality of locations for the time period is determined for each location of the plurality of locations. As shown at procedure 730, a variance of the air quality information for each location of the plurality of locations for the time period is determined. In some embodiments, as shown at procedure 735, a logarithm of the variance of the air quality information for each location of the plurality of locations for the time period is determined.

At procedure 740, the plurality of locations is evaluated according to the severe air quality percentile for the air quality information and the variance of the air quality information for each location. In some embodiments, as shown at procedure 750, a maximum/minimum normalization operation is performed on the severe air quality percentile for the air quality information and the variance of the air quality information for each location of the plurality of locations. In some embodiments, as shown at procedure 760, a clustering operation is performed on the severe air quality percentile for the air quality information and the variance of the air quality information for each location of the plurality of locations. For example, the clustering operation can determine five clusters of locations that are indicative of the relative air quality for the clustered locations (e.g., air quality that is great, good, average, poor, very poor). In some embodiments, as shown at procedure 770, a visualization based at least in part on the evaluation of the plurality of locations according to the severe air quality percentile for the air quality information and the variance of the air quality information is displayed.

It is noted that any of the procedures, stated above, regarding flow diagram 700 of FIG. 7 may be implemented in hardware, or a combination of hardware with firmware and/or software. For example, any of the procedures are implemented by a processor(s) of a cloud environment and/or a computing environment.

One or more embodiments of the present invention may be implemented as one or more computer programs or as one or more computer program modules embodied in one or more computer readable media. The term computer readable medium refers to any data storage device that can store data which can thereafter be input to a computer system—computer readable media may be based on any existing or subsequently developed technology for embodying computer programs in a manner that enables them to be read by a computer. Examples of a computer readable medium include a hard drive, network attached storage (NAS), read-only memory, random-access memory (e.g., a flash memory device), a CD (Compact Discs)—CD-ROM, a CD-R, or a CD-RW, a DVD (Digital Versatile Disc), a magnetic tape, and other optical and non-optical data storage devices. The computer readable medium can also be distributed over a network coupled computer system so that the computer readable code is stored and executed in a distributed fashion.

### CONCLUSION

Although one or more embodiments of the present invention have been described in some detail for clarity of understanding, it will be apparent that certain changes and modifications may be made within the scope of the claims. Accordingly, the described embodiments are to be considered as illustrative and not restrictive, and the scope of the claims is not to be limited to details given herein, but may be modified within the scope and equivalents of the claims. The description as set forth is not intended to be exhaustive or to limit the embodiments to the precise form disclosed. Rather, the specific features and acts described above are disclosed as example forms of implementing the claims. In the claims, elements and/or steps do not imply any particular order of operation, unless explicitly stated in the claims.

Reference throughout this document to “one embodiment,” “certain embodiments,” “an embodiment,” “various embodiments,” “some embodiments,” or similar term means that a particular feature, structure, or characteristic described in connection with the embodiment is included in at least one embodiment. Thus, the appearances of such phrases in various places throughout this specification are not necessarily all referring to the same embodiment. Furthermore, the particular features, structures, or characteristics of any embodiment may be combined in any suitable manner with one or more other features, structures, or characteristics of one or more other embodiments without limitation.

Many variations, modifications, additions, and improvements are possible, regardless of the degree of virtualization. Plural instances may be provided for components, operations or structures described herein as a single instance. Finally, boundaries between various components, operations and data stores are somewhat arbitrary, and particular operations are illustrated in the context of specific illustrative configurations. Other allocations of functionality are envisioned and may fall within the scope of the invention(s). In general, structures and functionality presented as separate components in exemplary configurations may be implemented as a combined structure or component. Similarly, structures and functionality presented as a single component may be implemented as separate components. These and other variations, modifications, additions, and improvements may fall within the scope of the appended claims(s).

What is claimed is:

1. A computer-implemented method for evaluating air quality, the method comprising:
receiving air quality information for a plurality of locations for a plurality of days over a time period;
for each location of the plurality of locations:
determining a severe air quality percentile for the air quality information for each location of the plurality of locations for the time period; and
determining a variance of the air quality information for each location of the plurality of locations for the time period; and
evaluating the plurality of locations according to the severe air quality percentile for the air quality information and the variance of the air quality information.

2. The method of claim 1, wherein the evaluating the plurality of locations according to the severe air quality percentile for the air quality information and the variance of the air quality information comprises:
performing a clustering operation on the severe air quality percentile for the air quality information and the variance of the air quality information for each location of the plurality of locations.

3. The method of claim 2, wherein the evaluating the plurality of locations according to the severe air quality percentile for the air quality information and the variance of the air quality information further comprises:
performing a maximum/minimum normalization operation on the severe air quality percentile for the air quality information and the variance of the air quality information for each location of the plurality of locations.

4. The method of claim 2, further comprising:
displaying a visualization of output of the clustering operation.

5. The method of claim 2, wherein the clustering operation generates five clusters of locations of the plurality of locations.

6. The method of claim 1, wherein the severe air quality percentile is a 90^{th} percentile air quality index (AQI).

7. The method of claim 1, further comprising:
for each location of the plurality of locations:
determining a logarithm of the variance of the air quality information for each location of the plurality of locations for the time period.

8. The method of claim 1, wherein the air quality information received is a daily average air quality for a location.

9. The method of claim 8, wherein the time period is at least one year, and wherein the air quality information for at least half of the days of the time period is received for each location of the plurality of locations.

10. The method of claim 1, further comprising:
displaying a visualization based at least in part on the evaluating the plurality of locations according to the severe air quality percentile for the air quality information and the variance of the air quality information.

11. A non-transitory computer readable storage medium having computer readable program code stored thereon for causing a computer system to perform a method for evaluating air quality, the method comprising:
receiving air quality information for a plurality of locations for a plurality of days over a time period;
for each location of the plurality of locations:
determining a severe air quality percentile for the air quality information for each location of the plurality of locations for the time period;
determining a variance of the air quality information for each location of the plurality of locations for the time period; and
determining a logarithm of the variance of the air quality information for each location of the plurality of locations for the time period; and
evaluating the plurality of locations according to the severe air quality percentile for the air quality information and the variance of the air quality information, the evaluating comprising:
performing a maximum/minimum normalization operation on the severe air quality percentile for the air quality information and the logarithm of the variance of the air quality information for each location of the plurality of locations; and
performing a clustering operation on the severe air quality percentile for the air quality information and the logarithm of the variance of the air quality information for each location of the plurality of locations.

12. The non-transitory computer readable storage medium of claim 11, the method further comprising:
displaying a visualization of output of the clustering operation.

13. The non-transitory computer readable storage medium of claim 11, wherein the clustering operation generates five clusters of locations of the plurality of locations.

14. The non-transitory computer readable storage medium of claim 11, wherein the severe air quality percentile is a 90^{th} percentile AQI.

15. The non-transitory computer readable storage medium of claim 11, wherein the air quality information received is a daily average air quality for a location.

16. The non-transitory computer readable storage medium of claim 15, wherein the time period is at least one year, and wherein the air quality information for at least half of the days of the time period is received for each location of the plurality of locations.

17. A system for transformation of evaluating air quality, the system comprising:
a memory device; and
a hardware processor coupled with memory device, the hardware processor configured to:
receive air quality information for a plurality of locations for a plurality of days over a time period, wherein the air quality information received is a daily average air quality for each location, wherein the time period is at least one year, and wherein the air quality information for at least half of the days of the time period is received for each location of the plurality of locations;
for each location of the plurality of locations:
determine a severe air quality percentile for the air quality information for each location of the plurality of locations for the time period;
determine a variance of the air quality information for each location of the plurality of locations for the time period; and
determine a logarithm of the variance of the air quality information for each location of the plurality of locations for the time period; and
perform a maximum/minimum normalization operation on the severe air quality percentile for the air quality information and the logarithm of the variance of the air quality information for each location of the plurality of locations; and
perform a clustering operation on the severe air quality percentile for the air quality information and the logarithm of the variance of the air quality information for each location of the plurality of locations.

18. The system of claim 17, wherein the hardware processor further configured to:
display a visualization of output of the clustering operation.

19. The system of claim 17, wherein the clustering operation generates five clusters of locations of the plurality of locations.

20. The system of claim 17, wherein the severe air quality percentile is a 90^{th} percentile AQI.
